(19) United States
(12) Patent Application Publication
(10) Pub. No.: US 2024/0215929 A1
(43) Pub. Date: Jul. 4, 2024

(54) DISPLAY DATA GENERATION DEVICE, DISPLAY DATA GENERATION METHOD, AND NON-TRANSITORY COMPUTER-READABLE STORAGE MEDIUM

(71) Applicant: Nihon Kohden Corporation, Tokyo (JP)

(72) Inventor: Daichi Yamakawa, Tokorozawa-shi (JP)

(21) Appl. No.: 18/397,890

(22) Filed: Dec. 27, 2023

(30) Foreign Application Priority Data
Dec. 28, 2022 (JP) 2022-211970

(51) Int. Cl. A61B 5/00; A61B 90/92

(52) U.S. Cl. CPC A61B 5/743; A61B 5/7445; A61B 90/92

(57) ABSTRACT

A display data generation device includes an input interface configured to receive vital signs information of a subject, one or more processors, and one or more memories configured to store at least one instruction executable by the processor. When the at least one command is executed by the processor, the display data generation device generates display data, the display data including the vital signs information and background information that indicates a background corresponding to a condition of the subject determined based on the vital signs information and a determination method for determining the condition of the subject, the background information overlapping with the vital signs information.

### CROSS-REFERENCE TO RELATED APPLICATIONS

The present application claims the benefit of priority of Japanese Patent Application No. 2022-211970, filed on Dec. 28, 2022, the content of which is incorporated herein by reference.

### TECHNICAL FIELD

The presently disclosed subject matter relates to a display data generation device, a display data generation method, and a non-transitory computer-readable storage medium.

### BACKGROUND ART

JP2021-121368A discloses a patient monitor that displays, among trend graphs of alarm setting values related to a plurality of pieces of vital signs information, a trend graph of an alarm setting value related to vital signs information selected by a user on the same screen as a trend graph of vital signs information.

### SUMMARY

There is a need to check vital signs information of a subject and condition information indicating a condition of the subject at a glance. At this time, it is desired that a condition level of the subject indicated by the condition information can also be checked at a glance. For example, in the patient monitor according to JP2021-121368A, a trend graph of vital signs information and a trend graph of an alarm setting value can be checked on the same screen, but a location where an alarm occurs can be recognized only in the trend graph of the alarm setting value. In this respect, there is room for improvement in the patient monitor according to JP2021-121368A.

An object of the presently disclosed subject matter is to provide a display data generation device, a display data generation method, and a non-transitory computer-readable storage medium that allow a user to check vital signs information of a subject and information indicating a condition of the subject at a glance.

According to a first aspect of the presently disclosed subject matter, there is provided a display data generation device including:
an input interface configured to receive vital signs information of a subject;
one or more processors; and
one or more memories configured to store at least one instruction executable by the processor, wherein
when the at least one command is executed by the processor, the display data generation device generates display data, the display data including the vital signs information and background information that indicates a background corresponding to a condition of the subject determined based on the vital signs information and a determination method for determining the condition of the subject, the background information overlapping with the vital signs information.

According to a second aspect of the presently disclosed subject matter, there is provided a display data generation method executed by a display data generation device, the method including:
generating display data, the display data including vital signs information of a subject and background information that indicates a background corresponding to a condition of the subject determined based on the vital signs information and a determination method for determining the condition of the subject, the background information overlapping with the vital signs information.

According to a third aspect of the presently disclosed subject matter, there is provided a non-transitory computer-readable storage medium storing a computer program executable by one or more processors provided in a display data generation device, when being executed, the computer program causing the display data generation device to
generate display data, the display data including vital signs information of a subject and background information that indicates a background corresponding to a condition of the subject determined based on the vital signs information and a determination method for determining the condition of the subject, the background information overlapping with the vital signs information.

According to the display data generation device having the above configuration, display data including vital signs information and background information is generated, in which the background information overlaps with the vital signs information. The background information indicates the background corresponding to the condition of the subject, and is thus information indicating the condition of the subject. Accordingly, the display data generated by the display data generation device having the above configuration can include not only the vital signs information but also the background information that indicates the condition of the subject and overlaps with the vital signs information. For this reason, the user can visually recognize the vital signs information and the background information overlapping with the vital signs information at the same time, so that the condition of the subject can be checked at a glance.

According to the display data generation device having the above configuration, the vital signs information of the subject and the information indicating the condition of the subject can be checked at a glance. A display data generation method, and a non-transitory computer-readable storage medium according to the above configuration can also implement the same or similar effect.

According to the presently disclosed subject matter, it is possible to provide a display data generation device, a display data generation method, and a non-transitory computer-readable storage medium that allow a user to check vital signs information of a subject and information indicating a condition of the subject at a glance.

### BRIEF DESCRIPTION OF DRAWINGS

FIG. 1 is a block diagram of a display data generation system according to an embodiment of the presently disclosed subject matter;

FIG. 2 illustrates reference values of qSOFA that is one of determination methods;

FIG. 3 illustrates reference values of NEWS that is one of determination methods;

FIG. 4 is a flowchart of a display data generation method according to an embodiment of the presently disclosed subject matter;

FIG. 5 illustrates an example of a screen on which display data is displayed;

FIG. 6 illustrates an example of the screen on which display data is displayed;

FIG. 7 illustrates an example of the screen on which display data is displayed; and

FIG. 8 illustrates an example of the screen on which display data is displayed.

### DESCRIPTION OF EMBODIMENTS

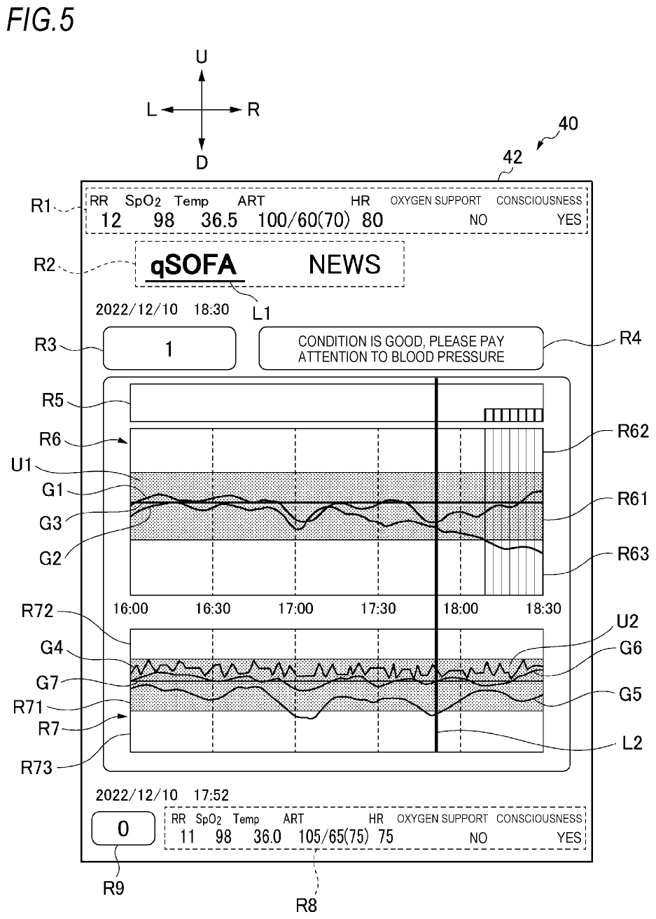

Hereinafter, an example of an embodiment of the presently disclosed subject matter will be described with reference to the drawings. In the description of the present embodiment, a “left-right direction” and an “upward-downward direction” may be appropriately referred to for convenience of description. These directions are relative directions set for a display 42 of a user terminal device 40 illustrated in FIGS. 5 to 8. Here, the “left-right direction” can include a “leftward direction” and a “rightward direction”. The “upward-downward direction” can include an “upward direction” and a “downward direction”. In each of the drawings, a reference sign U indicates the upward direction. A reference sign D indicates the downward direction. A reference sign L indicates the leftward direction. A reference sign R indicates the rightward direction.

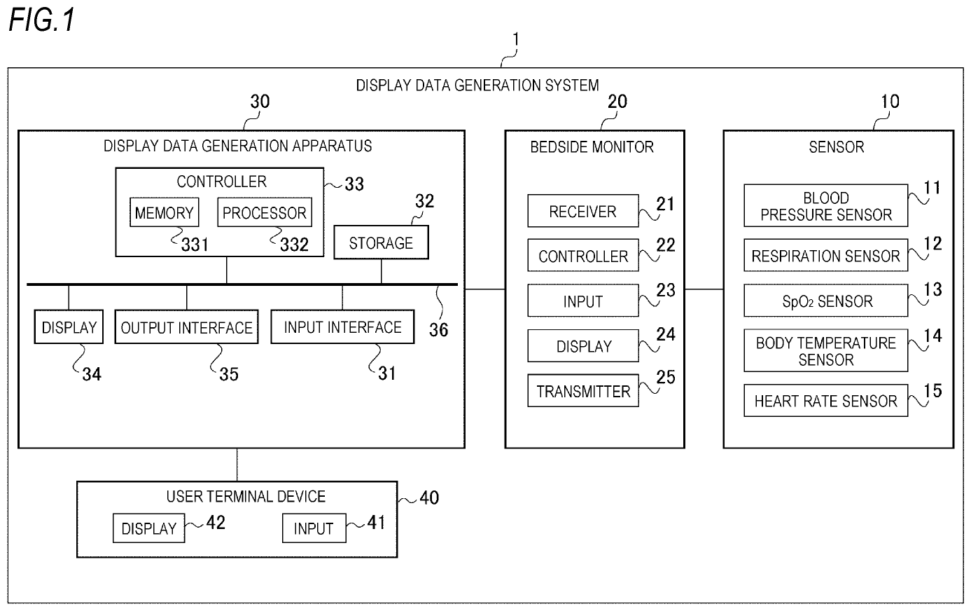

First, a display data generation system 1 according to the present embodiment will be described with reference to FIG. 1. The display data generation system 1 is used in a medical facility such as a hospital. As illustrated in FIG. 1, the display data generation system 1 can include a sensor 10, a bedside monitor 20, a display data generation device 30, and the user terminal device 40.

The sensor 10 can include a blood pressure sensor 11, a respiration sensor 12, an SpO₂ sensor 13, a body temperature sensor 14, and a heart rate sensor 15. The sensor 10 may further include another sensor different from these sensors. The blood pressure sensor 11 obtains a blood pressure signal (an example of a vital signs signal) indicating a temporal change in blood pressure (systolic blood pressure, diastolic blood pressure, and mean blood pressure) of a subject. The blood pressure sensor 11 is, for example, a blood pressure monitor or a catheter that obtains a vital blood pressure value. The respiration sensor 12 obtains a respiratory signal (an example of a vital signs signal) such as a respiratory rate indicating a respiratory state of the subject. The respiration sensor 12 is, for example, an electrode that obtains an impedance respiratory rate. The SpO₂ sensor 13 obtains an SpO₂ signal (an example of a vital signs signal) indicating a transcutaneous arterial oxygen saturation (SpO₂) of the subject. The SpO₂ sensor 13 is, for example, a probe that obtains SpO₂. The body temperature sensor 14 obtains a body temperature signal (an example of a vital signs signal) indicating a body temperature of the subject. The body temperature sensor 14 is a body temperature meter that measures the body temperature of the subject, a body temperature probe connected to the body temperature meter, or the like. The heart rate sensor 15 obtains a heart rate signal (an example of a vital signs signal) indicating a heart rate of the subject. The heart rate sensor 15 is, for example, an electrode that obtains a heart rate. In the present embodiment, the sensor 10 obtains a vital signs signal by a passive method, and may also obtain a vital signs signal by an active method. In the measurement of the vital signs signal by the passive method, the vital signs signal is measured by receiving a signal from the object without transmitting a signal such as light or an electromagnetic wave from the sensor 10 or the like to the object. In the measurement of the vital signs signal by the active method, the vital signs signal information is measured by transmitting a signal from the sensor 10 or the like to the object and receiving a signal reflected by or transmitted through the object.

The bedside monitor 20 is disposed, for example, in a vicinity of the subject wearing the sensor 10. The bedside monitor 20 is electrically connected to the sensor 10 and the display data generation device 30 in a wired or wireless manner. The bedside monitor 20 can include a receiver 21, a controller 22, an input 23, a display 24, and a transmitter 25.

The receiver 21 receives various vital signs signals obtained by the sensor 10. The receiver 21 transmits the received various vital signs signals to the controller 22.

The controller 22 has a hardware configuration and can include a memory and a processor. For example, the memory can include a read only memory (ROM) that stores various computer programs, and a random access memory (RAM) having a plurality of work areas in which various computer programs to be executed by the processor are stored. The processor is, for example, a central processing unit (CPU), loads to the RAM a computer program specified from various computer programs incorporated in the ROM, and executes various processes in cooperation with the RAM. For example, the controller 22 generates various pieces of vital signs information based on various vital signs signals by executing computer programs by the processor in cooperation with the RAM. Examples of the vital signs information can include blood pressure information based on the blood pressure signal, respiratory information based on the respiratory signal, SpO₂ information based on the SpO₂ signal, body temperature information based on the body temperature signal, and heart rate information based on the heart rate signal. The controller 22 transmits various pieces of vital signs information to the input 23.

The input 23 receives an input operation by a user such as a medical worker. The user is a medical worker such as a doctor or a nurse. The input 23 is, for example, a touch panel on the display 24, and an operation button attached to a case. The user inputs, for example, consciousness information (an example of vital signs information) related to the consciousness of the subject and oxygen support information (an example of vital signs information) related to the necessity of oxygen support to the input 23. For example, when the user confirms that the subject is not conscious, the user inputs consciousness information indicating that the subject is not conscious to the input 23. For example, when the user determines that it is necessary to perform oxygen support to the subject, the user inputs oxygen support information indicating that it is necessary to perform oxygen support to the subject to the input 23. In this way, the bedside monitor 20 can obtain not only vital signs information based on a vital signs signal of the subject obtained by the sensor 10 but also vital signs information of the subject via the input 23.

The display 24 is, for example, a liquid crystal display. The display 24 displays, for example, various pieces of vital signs information generated by the controller 22 and various pieces of vital signs information input to the input 23.

The transmitter 25 transmits the various pieces of vital signs information generated by the controller 22 and the various pieces of vital signs information input to the input 23 to the display data generation device 30.

The display data generation device 30 can include an input interface 31, a storage 32, a controller 33, a display 34, and an output interface 35. These components are communicably connected to each other via a bus 36.

The input interface 31 receives various pieces of vital signs information transmitted from the bedside monitor 20. That is, the vital signs information on the subject is input to the input interface 31. The input interface 31 transmits, for example, the received vital signs information to the controller 33.

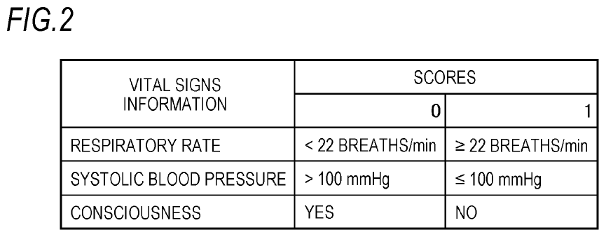

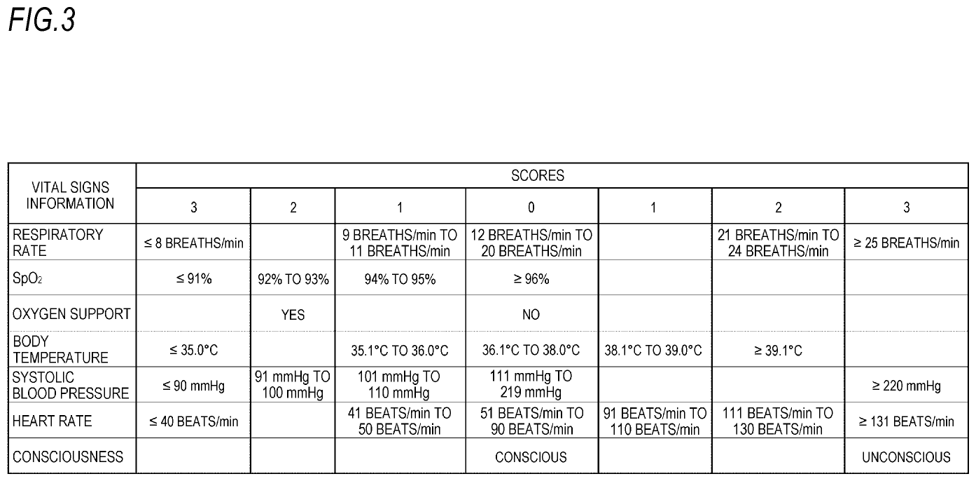

The storage 32 is a storage such as a hard disk drive (HDD), a solid state drive (SSD), or a flash memory. The storage 32 stores, for example, reference information indicating a reference value or the like of vital signs information used in quick SOFA (qSOFA) illustrated in FIG. 2, and type information indicating a type of vital signs information used in qSOFA. The storage 32 stores, for example, reference information indicating a reference value or the like of vital signs information used in National Early Warning Score (NEWS) illustrated in FIG. 3, and type information indicating a type of vital signs information used in NEWS. NEWS and qSOFA are determination methods used to determine whether the subject has sepsis. That is, the storage 32 stores determination methods for determining a condition of the subject.

As illustrated in FIG. 2, the vital signs information used in qSOFA is the respiratory rate, the systolic blood pressure, and the consciousness. When the respiratory rate of the subject per minute is 22 breaths or more, a score related to the respiratory rate is 1. On the other hand, when the respiratory rate of the subject per minute is less than 22 breaths, the score related to the respiratory rate is 0. When the systolic blood pressure of the subject is 100 mmHg or less, a score related to the systolic blood pressure is 1. On the other hand, when the systolic blood pressure of the subject exceeds 100 mmHg, the score related to the systolic blood pressure is 0. When the subject is not conscious, a score related to the consciousness is 1. On the other hand, when the subject is conscious, the score related to the consciousness is 0. These scores are summed up, and when the summed score is 2 or more, there is a high probability that the subject has sepsis (that is, the condition of the subject is poor). In the present embodiment, regarding the vital signs information illustrated in FIG. 2, a range in which the score is 0 is assumed to be a normal range of the vital signs information used for qSOFA, and a range other than the normal range (a range in which the score is 1) is assumed to be an abnormal range of the vital signs information used for qSOFA. In the present embodiment, a range in which the respiratory rate per minute of the subject is 0 breaths or more and less than 22 breaths is assumed to be a normal range of the respiratory information (respiratory rate) in qSOFA, and a range in which the respiratory rate per minute of the subject is 22 breaths or more is assumed to be an abnormal range of the respiratory information in qSOFA. A range in which the systolic blood pressure of the subject is more than 100 mmHg and 300 mmHg or less is assumed to be a normal range of the blood pressure information (systolic blood pressure) of qSOFA, and a range in which the systolic blood pressure of the subject is 100 mmHg or less is assumed to be an abnormal range of the blood pressure information in qSOFA. An upper limit value set in the normal range of the blood pressure information (systolic blood pressure) is, for example, a maximum value of the systolic blood pressure obtained from the subject or a value freely set by the user. Regarding the consciousness, it is assumed that the consciousness is digitized as 3 and unconsciousness is digitized as 1, a normal range of the consciousness information in qSOFA is 2 or more and 4 or less, and an abnormal range of the consciousness information in qSOFA is 0 or more and less than 2.

As illustrated in FIG. 3, the vital signs information used in NEWS can include the respiratory rate, the transcutaneous arterial oxygen saturation (SpO₂), the oxygen support, the body temperature, the systolic blood pressure, the heart rate, and the consciousness. For example, when the respiratory rate of a subject per minute is 8 breaths or less, or 25 breaths or more, a score related to the respiratory rate is 3. When the respiratory rate is 21 breaths or more and 24 breaths or less, the score related to the respiratory rate is 2. When the respiratory rate is 9 breaths or more and 11 breaths or less, the score related to the respiratory rate is 1. When the respiratory rate is 12 breaths or more and 20 breaths or less, the score related to the respiratory rate is 0. For example, when the subject is not conscious, a score related to the consciousness is 3. On the other hand, when the subject is conscious, the score related to the consciousness is 0. For the remaining parameters, a score is calculated based on a reference value or the like determined by a medical guideline or the like. These calculated scores are summed up. When the added score is 5 or more, or when there is one or more pieces of vital signs information having a score of 3 or more, there is a high probability that the subject has sepsis (that is, the condition of the subject is poor). In the present embodiment, regarding the vital signs information illustrated in FIG. 3, a range in which the score is 0 is assumed to be a normal range of the vital signs information used for NEWS, and a range other than the normal range (a range in which the score is 1 to 3) is assumed to be an abnormal range of the vital signs information used for NEWS. For example, a range in which the respiratory rate of the subject per minute is 12 breaths or more and 20 breaths or less is a normal range of the respiratory information (respiratory rate) in NEWS, and a range in which the respiratory rate of the subject per minute is 11 breaths or less or 21 breaths or more is an abnormal range of the respiratory information in NEWS. In the present embodiment, it is assumed that a range in which the transcutaneous arterial oxygen saturation of the subject is 96% or more and 100% or less is a normal range of the SpO₂ information in NEWS, and a range in which the transcutaneous arterial oxygen saturation of the subject is less than 96% is an abnormal range of the respiratory information in NEWS. Regarding the consciousness, it is assumed that the consciousness is digitized as 3 and the unconsciousness is digitized as 1, a normal range of the consciousness information in NEWS is 2 or more and 4 or less, and an abnormal range of the consciousness information in NEWS is 0 or more and less than 2. Regarding the oxygen support, it is assumed that a presence of oxygen support is digitized as 1, an absence of oxygen support is digitized as 3, a normal range of the oxygen support information in NEWS is 2 or more and 4 or less, and an abnormal range of the oxygen support information in NEWS is 0 or more and less than 2.

Description is referred back to FIG. 1, and the storage 32 will be described. The storage 32 stores various types of information such as normal range information indicating a normal range of the vital signs information set for each piece of vital signs information, abnormal range information indicating an abnormal range of the vital signs information set for each piece of vital signs information, and candidate determination method information indicating a determination method that is selectable but not used. For example, the storage 32 transmits the normal range information and the abnormal range information to the controller 33 based on a control signal from the controller 33.

The controller 33 has a hardware configuration and can include a memory 331 and a processor 332. For example, the memory 331 can include a read only memory (ROM) that stores various computer programs, and a random access memory (RAM) having a plurality of work areas in which various computer programs to be executed by the processor are stored. The processor 332 is, for example, a central processing unit (CPU), loads to the RAM a computer program specified from various computer programs incorporated in the ROM, and executes various processes in cooperation with the RAM. For example, the controller 33 implements various processes by executing computer programs by the processor 332 in cooperation with the RAM.

The controller 33 generates condition information indicating the condition of the subject based on the vital signs information received from the input interface 31 and the determination method stored in the storage 32. When the condition information is generated, the controller 33 generates background information indicating a background corresponding to the condition of the subject. The controller 33 generates display data including the vital signs information received from the input interface 31 and the background information. The display data may include not only the vital signs information and the background information but also other information such as the normal range information, the abnormal range information, the condition information, the determination method information indicating a used determination method, the candidate determination method information, and identification information described later. In the display data, the background information overlaps with the vital signs information. On the other hand, the condition information does not overlap with the vital signs information. The display data can include a first region in which vital signs information used in a determination method for determining the condition of the subject is displayed, a second region in which vital signs information not used in the determination method is displayed, and a third region in which vital signs information obtained at a specific time is displayed.

The controller 33 can control a color tone related to a display mode of the background information to be different from a color tone related to a display mode of the vital signs information. In the present embodiment, a color related to the display mode of the background information is lighter than a color related to the display mode of the vital signs information. For example, the controller 33 controls the color related to the display mode of the background information to be lighter than the color related to the display mode of the vital signs information by controlling the brightness and the chroma related to the display mode of the background information to be lower than the brightness and the chroma related to the display mode of the vital signs information. For example, the controller 33 controls the color related to the display mode of the background information to be lighter than the color related to the display mode of the vital signs information by controlling the brightness related to the display mode of the background information to be lower than the brightness related to the display mode of the vital signs information. For example, the controller 33 controls the color related to the display mode of the background information to be lighter than the color related to the display mode of the vital signs information by controlling the chroma related to the display mode of the background information to be lower than the chroma related to the display mode of the vital signs information. In the present embodiment, the color tone is determined by controlling RGB gradient by the controller 33. Here, the brightness can be obtained based on a maximum value of RGB, and the chroma is obtained based on a value obtained by dividing, by the maximum value of RGB, a value obtained by subtracting a minimum value of RGB from the maximum value of RGB. Accordingly, the brightness related to the display mode of the background information being lower than the brightness related to the display mode of the vital signs information means that a maximum value of RGB related to the display mode of the background information is smaller than a maximum value of RGB related to the display mode of the vital signs information. The chroma related to the display mode of the background information being lower than the chroma related to the display mode of the vital signs information means that a value obtained by dividing, by the maximum value of RGB, a value obtained by subtracting a minimum value of RGB from the maximum value of RGB related to the display mode of the background information is smaller than a value obtained by dividing, by the maximum value of RGB, a value obtained by subtracting a minimum value of RGB from the maximum value of RGB related to the display mode of the vital signs information.

The controller 33 can control a color tone related to a display mode of the condition information to be different from the color tone related to the display mode of the background information. In the present embodiment, the hue related to the display mode of the condition information corresponds to the hue related to the display mode of the background information. The expression “the hues correspond to each other” means that values obtained from the following formulas are the same. When a maximum value of RGB is MAX, a minimum value is MIN and R is the maximum value, a hue H=60×((G−B)/(MAX−MIN)). When G is the maximum value, the hue H=60×((B−R)/(MAX−MIN))+120.

When B is the maximum value, the hue H=60×((R−G)/(MAX−MIN))+240.

When RGB has the same value, the hue H is 0.

The color related to the display mode of the condition information is darker than the color related to the display mode of the background information. The color tone related to the display mode of the condition information and the color tone related to the display mode of the background information are also determined by controlling the RGB gradient by the controller 33 as described above.

The controller 33 generates a control signal for transmitting the normal range information and the abnormal range information corresponding to the vital signs information received from the input interface 31 to the controller 33, and transmits the control signal to the storage 32. The storage 32 transmits the normal range information and the abnormal range information corresponding to the vital signs information received from the input interface 31 to the controller 33 based on the control signal. In this manner, the controller 33 receives, from the storage 32, the normal range information and the abnormal range information corresponding to the vital signs information received from the input interface 31. The display data generated by the controller 33 may include the normal range information and the abnormal range information related to the vital signs information received from the input interface 31. In this case, the vital signs information overlaps with the background information and at least one of the normal range information and the abnormal range information. The controller 33 may generate the display data such that a color tone related to a display mode of the normal range information is different from a color tone related to a display mode of the abnormal range information. The color tone is also determined by controlling the RGB gradation by the controller 33 as described above.

The controller 33 classifies the vital signs information input to the input interface 31 into vital signs information used for a determination method and vital signs information not used for the determination method based on the determination method information and the type information. The controller 33 generates the identification information for identifying a determination method used and a determination method not used based on the determination method information and the candidate determination method information.

When a plurality of pieces of vital signs information are input to the input interface 31, the controller 33 generates unified range information indicating a unified range based on the normal range information and the abnormal range information corresponding to each piece of vital signs information. The unified range is a range for uniformly defining a normal range and an abnormal range of the plurality of pieces of vital signs information. The plurality of vital signs information input to the input interface 31 are collectively displayed in the unified range in the display data generated by the controller 33.

Computer programs may be stored in, for example, various types of non-transitory computer-readable storage media and supplied to a computer. The non-transitory computer-readable storage media can include various types of tangible storage media. Examples of the non-transitory computer-readable storage media can include a magnetic recording medium (for example, a flexible disk, a magnetic tape, or a hard disk drive), a magneto-optical recording medium (for example, a magneto-optical disk), a CD read only memory (ROM), a CD-R, a CD-R/W, and a semiconductor memory (for example, a mask ROM, a programmable ROM (PROM), an erasable PROM (EPROM), or a flash ROM).

The display 34 is a touch screen display such as a liquid crystal display or an organic EL display. The display 34 displays, for example, the display data received from the controller 33.

The output interface 35 outputs the display data received from the controller 33 to the user terminal device 40. For example, the output interface 35 outputs an output signal OS corresponding to the display data to the user terminal device 40. The output interface 35 may include, as necessary, a circuit that converts output data into the output signal OS that can be processed by the user terminal device 40.

The user terminal device 40 is an electronic device such as a desktop PC, a notebook PC, a tablet terminal, or a smartphone. The user terminal device 40 can include an input 41 and the display 42.

The input 41 receives an input operation by a user such as a medical worker. The input 41 is, for example, a touch panel on the display 42 or an operation button attached to a case. To the input 41, the determination method information indicating a used determination method, the selection time information indicating a specific time at which the user desires to check the vital signs information, and the like are input by the user. The input 41 transmits the determination method information, the selection time information, and the like input by the user to the display data generation device 30.

The display 42 is, for example, a liquid crystal display. The display 42 displays, for example, display data output from the output interface 35.

### First Embodiment

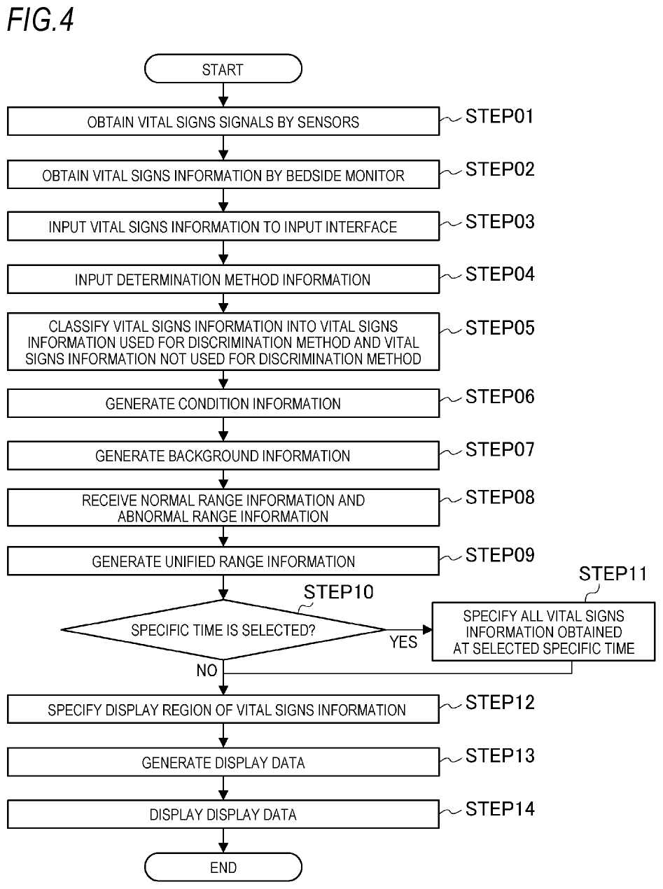

Next, a display data generation method according to the present embodiment will be described with reference to FIGS. 4 to 6. FIG. 4 is a flowchart of the display data generation method according to the present embodiment. It is assumed that a current time in the present embodiment is 18:30, Dec. 10, 2022, and the display data generation method illustrated in FIG. 4 is executed at 18:30, Dec. 10, 2022. In the present embodiment, it is assumed that the vital signs information is obtained from 16:00, Dec. 10, 2022 to 18:30, Dec. 10, 2022. In the present embodiment, it is assumed that the selection time information, which indicates that the time at which the user desires to check the vital signs information is 17:52, Dec. 10, 2022, is input to the input 41 of the user terminal device 40. That is, the specific time selected by the user in the present embodiment is 17:52, Dec. 10, 2022.

In the present embodiment, the respiratory rate, the transcutaneous arterial oxygen saturation, the body temperature, the systolic blood pressure, the diastolic blood pressure, the mean blood pressure, the heart rate, the oxygen support, the consciousness, scores based on qSOFA, and scores based on NEWS of the subject at 17:52 and 18:30, Dec. 10, 2022 are as illustrated in Table 1.

| | | Transcutaneous | | Systolic | Diastolic | Mean |
|---|---|---|---|---|---|---|
| | Respiratory | arterial oxygen | Body | blood | blood | blood |
| Time | rate | saturation | temperature | pressure | pressure | pressure |
| 17:52, Dec.10, 2022 | 11 Breaths/min | 98% | 36.0° C. | 105 mmHg | 65 mmHg | 75 mmHg |
| 18:30, Dec.10, 2022 | 12 Breaths/min | 98% | 36.5° C. | 100 mmHg | 60 mmHg | 70 mmHg |

| | | | | Score | Score |
|---|---|---|---|---|---|
| | | Oxygen | | based on | based on |
| Time | Heart rate | support | Consciousness | qSOFA | NEWS |
| 17:52, Dec.10, 2022 | 75 Beats/min | No | Yes | 0 | 3 |
| 18:30, Dec.10, 2022 | 80 Beats/min | No | Yes | 1 | 2 |

As illustrated in FIG. 4, vital signs signals of the subject are obtained by the sensor 10 (STEP 01). In the present embodiment, the sensor 10 obtains the respiratory signal, the SpO₂ signal, the body temperature signal, the blood pressure signal, and the heart rate signal. The various vital signs signals obtained by the sensor 10 are transmitted to the receiver 21 of the bedside monitor 20.

The bedside monitor 20 obtains various pieces of vital signs information (STEP 02). In the present embodiment, the user inputs the oxygen support information and the consciousness information to the input 23. For this reason, the bedside monitor 20 obtains the respiratory information, the SpO₂ information, the body temperature information, the blood pressure information, and the heart rate information generated by the controller 22 based on the respiratory signal, the SpO₂ signal, the body temperature signal, the blood pressure signal, and the heart rate signal received via the receiver 21. The bedside monitor 20 obtains the oxygen support information and the consciousness information via the input 23. The transmitter 25 of the bedside monitor 20 transmits various pieces of vital signs information generated by the controller 22 and various pieces of vital signs information input to the input 23 to the input interface 31 of the display data generation device 30.

Various pieces of vital signs information transmitted from the transmitter 25 of the bedside monitor 20 is input to the input interface 31 (STEP 03). The input interface 31 transmits the input vital signs information to the controller 33.

The determination method information indicating a used determination method is input to the input 41 of the user terminal device 40 (STEP 04). For example, the user inputs determination method information indicating that the used determination method is qSOFA to the input 41 of the user terminal device 40. The input of the determination method information to the input 41 is performed, for example, by typing the used determination method on the touch panel on the display 42. The user terminal device 40 transmits the determination method information input to the input 41 to the display data generation device 30.

The controller 33 classifies the vital signs information input to the input interface 31 into vital signs information used for the determination method and vital signs information not used for the determination method based on the determination method information received from the user terminal device 40 and the type information stored in the storage 32 (STEP 05). When the used determination method is qSOFA, for example, the vital signs information used in the determination method is the respiratory information, the blood pressure information, and the consciousness information, and the vital signs information not used in the determination method is the SpO₂ information, the body temperature information, the heart rate information, and the oxygen support information. In STEP 05, the controller 33 generates the identification information based on the determination method information and the candidate determination method information.

The controller 33 generates the condition information indicating the condition of the subject based on the vital signs information received from the input interface 31 and the reference information stored in the storage 32 (STEP 06). When the used determination method is qSOFA, the controller 33 generates the condition information based on the respiratory information, the blood pressure information, and the consciousness information, and the reference information related to qSOFA. Here, as an example, a case in which the controller 33 generates the condition information indicating the condition of the subject at the current time will be described. In the present embodiment, the respiratory rate of the subject at the current time is 12 breaths. The systolic blood pressure of the subject at the current time is 100. At the current time, the subject is conscious. Accordingly, the controller 33 generates condition information indicating that the score based on qSOFA at the current time is 1.

The controller 33 generates the background information indicating the background corresponding to the condition of the subject determined based on the vital signs information used in the determination method used for determining the condition of the subject and the determination method (STEP 07). For example, when the condition of the subject is determined based on qSOFA at the current time, the controller 33 generates the background information indicating the background according to the score (that is, 1) based on qSOFA at the current time.

The controller 33 generates a control signal for transmitting the normal range information and the abnormal range information corresponding to the vital signs information received from the input interface 31 to the controller 33, and transmits the control signal to the storage 32. Accordingly, the controller 33 receives the normal range information and the abnormal range information corresponding to the vital signs information (STEP 08).

The controller 33 generates the unified range information indicating a unified range based on the normal range information and the abnormal range information corresponding to the vital signs information (STEP 09). In the present embodiment, the controller 33 generates the unified range information indicating the unified range based on the normal range information and the abnormal range information corresponding to the vital signs information used in the determination method, and the unified range information indicating the unified range based on the normal range information and the abnormal range information corresponding to the vital signs information not used in the determination method.

First, the unified range information indicating the unified range based on the normal range information and the abnormal range information corresponding to the vital signs information used in the determination method will be described. Here, it is assumed that qSOFA is used as the determination method. The normal range of the respiratory rate per minute corresponding to the respiratory information is 0 breaths or more and less than 22 breaths, and the abnormal range of the respiratory rate per minute corresponding to the respiratory information is 22 breaths or more. The normal range of the systolic blood pressure corresponding to the blood pressure information is more than 100 mmHg and 300 mmHg or less, and the abnormal range of the systolic blood pressure corresponding to the blood pressure information is 100 mmHg or less. The normal range of the consciousness corresponding to the consciousness information is 2 or more and 4 or less, and the abnormal range of the consciousness corresponding to the consciousness information is 0 or more and less than 2. The controller 33 generates the unified range information indicating the unified range based on the normal range information and the abnormal range information corresponding to the respiratory information, the blood pressure information, and the consciousness information. The controller 33 calculates a median value of an upper limit value and a lower limit value in the normal range of each piece of the vital signs information, divides the upper limit value and the lower limit value in the normal range of each piece of the vital signs information by the median value corresponding to each piece of vital signs information, and then multiplies values after the division by a largest median value to generate the unified range information. That is, the controller 33 corrects normal ranges of the vital signs information, which are originally different numerical ranges, as described above, thereby matching display positions of the normal ranges of the vital signs information and setting the unified range. In the present embodiment, the median value of the upper limit value and the lower limit value in the normal range of the respiratory information is 11 breaths. The median value of the upper limit value and the lower limit value in the normal range of the blood pressure information is 200 mmHg. The median value of the upper limit value and the lower limit value in the normal range of the consciousness information is 3. The controller 33 multiplies the upper limit value and the lower limit value in the normal range of the respiratory information by 200/11 and multiplies the upper limit value and the lower limit value in the normal range of the consciousness information by 200/3 to generate the unified range information. At this time, the controller 33 multiplies the respiratory rate indicated by the obtained respiratory information by 200/11, and multiplies the numerical value corresponding to the obtained consciousness information by 200/3.

Next, the unified range information indicating the unified range based on the normal range information and the abnormal range information corresponding to the vital signs information not used in the determination method will be described. When qSOFA is used in the present embodiment, the normal range of the vital signs information (the SpO₂ information, the body temperature information, the heart rate information, and the oxygen support information) not used for qSOFA is in accordance with the normal range and the abnormal range of the vital signs information in NEWS. That is, for example, the normal range of the body temperature information is 36.1 degrees or more and 38.0 degrees or less, and the abnormal range of the body temperature information is 36.0 degrees or less or 38.1 degrees or more. According to the above-described principle, the controller 33 generates the unified range information indicating the unified range based on the normal range information and the abnormal range information corresponding to the SpO₂ information, the body temperature information, the heart rate information, and the oxygen support information.

As illustrated in FIG. 4, the controller 33 determines whether a specific time at which the vital signs information is desired to be checked is selected (STEP 10). When selecting the specific time at which the user desires to check the vital signs information, the user inputs the selection time information indicating the specific time at which the user desires to check the vital signs information to the input 41 of the user terminal device 40. When the selected time information is input to the input 41 of the user terminal device 40, the user terminal device 40 transmits the selected time information input to the input 41 to the display data generation device 30. When the display data generation device 30 receives the selection time information from the user terminal device 40, the controller 33 determines that the specific time at which the vital signs information is desired to be checked is selected (YES in STEP 10), and performs STEP 11. On the other hand, when the display data generation device 30 does not receive the selection time information from the user terminal device 40, the controller 33 determines that no specific time at which the vital signs information is desired to be checked is selected (NO in STEP 10), and performs STEP 12. In the present embodiment, since 17:52, Dec. 10, 2022 is selected as the specific time at which the vital signs information is desired to be checked (YES in STEP 10), the controller 33 performs STEP 11.

The controller 33 specifies all the vital signs information obtained at the specific time selected by the user (STEP 11). Since the specific time selected by the user in the present embodiment is 17:52, Dec. 10, 2022, all the vital signs information (the respiratory information, the SpO₂ information, the body temperature information, the blood pressure information, the heart rate information, the oxygen support information, and the consciousness information) obtained at 17:52, Dec. 10, 2022 is specified.

The controller 33 specifies the display region of the vital signs information (STEP 12). Specifically, the controller 33 specifies the first region as the display region of the vital signs information used for the determination method, the second region as the display region of the vital signs information not used for the determination method, and the third region as the display region of the vital signs information obtained at a specific time freely selected by the user. When the used determination method is qSOFA, for example, the display region of the respiratory information, the blood pressure information, and the consciousness information obtained from 16:00, Dec. 10, 2022 to the current time is the first region. The display region of the SpO₂ information, the body temperature information, the heart rate information, and the oxygen support information obtained from 16:00, Dec. 10, 2022 to the current time is the second region. The display region of the respiratory information, the SpO₂ information, the body temperature information, the blood pressure information, the heart rate information, the oxygen support information, and the consciousness information obtained at 17:52, Dec. 10, 2022 is the third region.

The controller 33 generates display data including the vital signs information, the background information, the normal range information, the abnormal range information, the condition information, the determination method information, the candidate determination method information, and the identification information (STEP 13). The controller 33 transmits the generated display data to the display 34 and/or the output interface 35. When the display data is transmitted to the output interface 35, the output interface 35 outputs the received display data to the user terminal device 40. In the present embodiment, the display data is transmitted to the output interface 35 and output to the user terminal device 40.

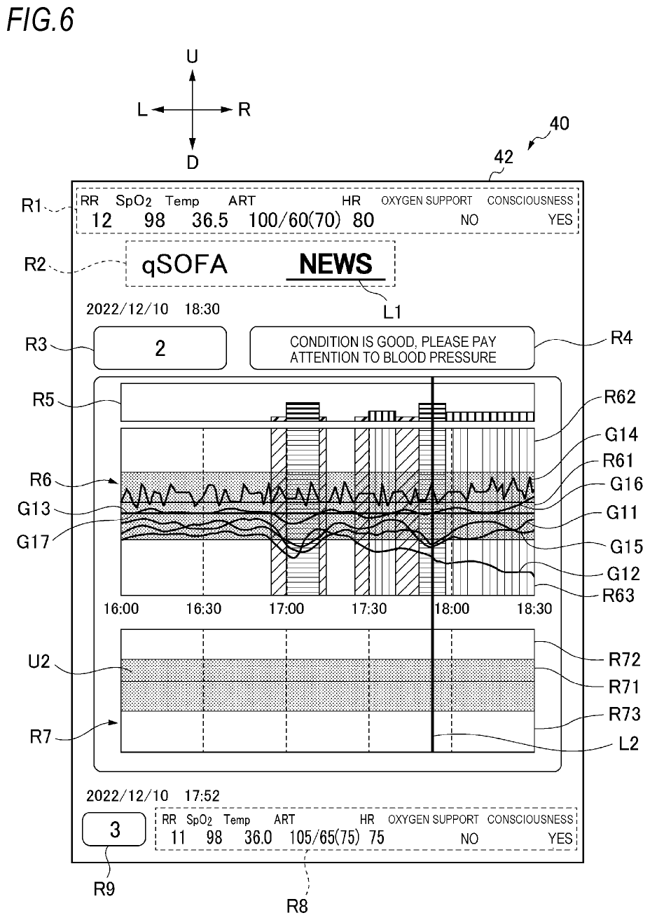

When the display data is output to the user terminal device 40, the display data is displayed on the display 42 of the user terminal device 40 (STEP 14). At this time, the display data illustrated in FIG. 5 or FIG. 6 is displayed on the display 42 of the user terminal device 40. The display data illustrated in FIG. 5 is display data displayed on the display 42 when qSOFA is used. The display data illustrated in FIG. 6 is display data displayed on the display 42 when NEWS is used.

First, display data displayed on the display 42 when qSOFA is used as the determination method will be described. As illustrated in FIG. 5, the display data can include regions R1 to R9. The region R1 displays the vital signs information (the respiratory information, the SPO₂ information, the body temperature information, the blood pressure information, the heart rate information, the oxygen support information, and the consciousness information) of the subject at the current time of 18:30, Dec. 10, 2022.

The region R2 below the region R1 displays a determination method for determining the condition of the subject. In the present embodiment, qSOFA and NEWS are displayed in the region R2. In the present embodiment, the used determination method is qSOFA, and thus qSOFA is the determination method indicated by the determination method information, and NEWS is the determination method indicated by the candidate determination method information. In a vicinity of characters of the used determination method indicated by the determination method information, the identification information for identifying the determination method used and the determination method not used is displayed. In the example illustrated in FIG. 5, qSOFA is used, and thus a straight line L1 is displayed in the vicinity of characters “qSOFA” as the identification information indicating that qSOFA is used. The straight line L1 is displayed below the characters “qSOFA”.

The region R3 below the region R2 displays, as the condition information based on the used determination method at 18:30, Dec. 10, 2022, the score based on the used determination method. In the example illustrated in FIG. 5, the used determination method is qSOFA, and the score based on qSOFA at 18:30, Dec. 10, 2022 is 1, and thus “1” is displayed in the region R3.

The region R4 on a right side of the region R3 displays a message corresponding to a determination result of the controller 33 at the current time. In the example illustrated in FIG. 5, a message “condition is good, please pay attention to blood pressure” is displayed in the region R4. The information displayed in the region R4 is not limited to a message, and for example, a color or the like corresponding to the determination result of the controller 33 at the current time may be displayed.

The region R5 below the region R3 and the region R4 displays the condition information based on the used determination method in a period from 16:00, Dec. 10, 2022 to the current time in a form of bar graphs. In the example illustrated in FIG. 5, bar graphs corresponding to scores based on qSOFA in the period from 16:00, Dec. 10, 2022 to the current time are displayed in the region R5. In the present embodiment, when the score of qSOFA is 1, the bar graph is colored in yellow, and when the score based on qSOFA is 2 or more, the bar graph is colored in red. In FIG. 5, a bar graph hatched with vertical lines indicates a bar graph colored in yellow. The bar graphs are arranged in time series, and the left bar graphs represent the condition information before that of the right bar graphs in time.

The region R6 below the region R5 displays, among the vital signs information from 16:00, Dec. 10, 2022 to the current time, the vital signs information used for the used determination method (the determination method indicated by the determination method information) in a form of vital graphs extending on a timeline. Accordingly, the region R6 is an example of the first region. In the example illustrated in FIG. 5, the vital signs information (the respiratory information, the blood pressure information, and the consciousness information) used in qSOFA from 16:00, Dec. 10, 2022 to the current time is displayed in the region R6 in a form of vital graphs extending on the timeline. The vital graph indicating the respiratory information is a graph G1. The vital graph indicating the blood pressure information is a graph G2. The vital graph indicating the consciousness information is a graph G3. The graphs G1 to G3 do not overlap with the bar graphs of the condition information displayed in the region R5. Timelines in the region R5 and the region R6 coincide with each other, and the graphs G1 to G3 and the bar graphs of the condition information are displayed in correspondence with each other along the timelines.

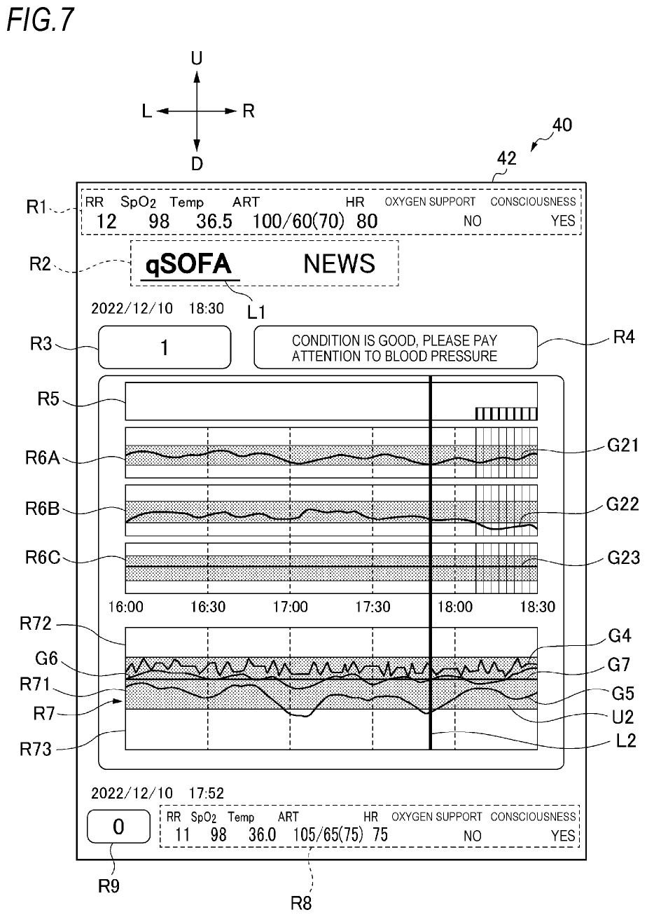

In the region R6, the background information indicating the background according to the condition of the subject determined based on the vital signs information and the determination method overlaps with the graphs G1 to G3. That is, the background information overlaps with the vital signs information. The background information in the region R6 coincides with the timeline of the condition information in the region R5. The background corresponding to the background information at a certain time is colored in a color that has a hue corresponding to a hue of a color of the bar graph of the condition information at the same time and is lighter than the color of the bar graph. In other words, at least one of the brightness and the chroma related to the display mode of the condition information is higher than at least one of the brightness and the chroma related to the display mode of the background information. The present specification is not intended to compare the brightness related to the display mode of the condition information and the chroma related to the display mode of the background information, or the chroma related to the display mode of the condition information and the brightness related to the display mode of the background information. In the example illustrated in FIG. 5, the background information at the time when the score of qSOFA is 1 is colored in light yellow. For convenience of description, in FIG. 5, a thickness of the hatched line represents a height of the brightness and/or the chroma (that is, a color intensity) of the color. The thicker the hatched line is, the higher the brightness and/or the chroma of the color is (that is, the darker the color is). This also applies to the display data illustrated in FIGS. 6 and 7. In this manner, the color tone related to the display mode of the background information is different from the color tone related to the display mode of the condition information. In the present embodiment, the color related to the display mode of the background information is lighter than colors related to display modes of the graphs G1 to G3. In other words, at least one of the brightness and the chroma related to the display mode of the background information is lower than at least one of the brightness and the chroma related to the display modes of the graphs G1 to G3. The present specification is not intended to compare the brightness related to the display mode of the background information and the chroma related to the display mode of the graphs G1 to G3, or the chroma related to the display mode of the background information and the brightness related to the display mode of the graphs G1 to G3. For convenience of description, in FIG. 5, the thickness of the line represents the height of the brightness and/or the chroma (that is, the color intensity) of the color. The thicker the line, the higher the brightness and/or the chroma of the color (i.e., the darker the color). That is, the color tone related to the display mode of the background information is different from the color tone related to the display modes of the graphs G1 to G3.

The region R6 can include a region R61 corresponding to the normal range of the vital signs information used in the used determination method, and a region R62 and a region R63 corresponding to the abnormal range of the vital signs information used in the used determination method. In the example illustrated in FIG. 5, the normal range and the abnormal range of the vital signs information used in the used determination method are uniformly defined by a unified range U1 indicated by the unified range information generated by the controller 33 according to the above principle. Specifically, in the region R6, a plurality of pieces of vital signs information (graphs G1 to G3) are collectively displayed in the unified range U1. That is, in the region R6, display positions of the normal ranges of the vital signs information used in the used determination method coincide with each other. In other words, in the region R6, the normal ranges of the vital signs information used in the used determination method coincide with a vertical axis (an axis extending in a vertical direction in FIG. 5) in the region R6. The graphs G1 to G3 overlap with the region R61 or the region R63. Accordingly, the vital signs information overlaps with at least one of the normal range information and the abnormal range information. The color tone of the region R61 is different from color tones of the regions R62 and R63. That is, the color tone related to the display mode of the normal range information is different from the color tone related to the display mode of the abnormal range information. In the present embodiment, the color tone of the region R61 is gray, and the color tone of the regions R62 and R63 is black. That is, the color related to the display mode of the normal range information is lighter than the color related to the display mode of the abnormal range information. In other words, at least one of the brightness and the chroma related to the display mode of the normal range information is lower than at least one of the brightness and the chroma related to the display mode of the abnormal range information. The present specification is not intended to compare the brightness related to the display mode of the normal range information and the chroma related to the display mode of the abnormal range information, or the chroma related to the display mode of the normal range information and the brightness related to the display mode of the abnormal range information. For convenience of description, in FIG. 5, the presence or absence of dot hatching illustrates that the color tone related to the display mode of the normal range information is different from the color tone related to the display mode of the abnormal range information. This also applies to the display data illustrated in FIGS. 6 and 7.

The region R7 below the region R6 displays, among the vital signs information from 16:00, Dec. 10, 2022 to the current time, the vital signs information not used for the used determination method in a form of vital graphs extending on a timeline. Accordingly, the region R7 is an example of the second region. In the present embodiment, the vital signs information (the SpO₂ information, the body temperature information, the heart rate information, and the oxygen support information) not used in qSOFA from 16:00, Dec. 10, 2022 to the current time is displayed in the region R7 in a form of vital graphs extending on the timeline. The vital graph indicating the SpO₂ information is a graph G4. The vital graph indicating the body temperature information is a graph G5. The vital graph indicating the heart rate information is a graph G6. The vital graph indicating the oxygen support information is a graph G7. Timelines in the region R6 and the region R7 coincide with each other. That is, the timelines in the region R5, the region R6, and the region R7 all coincide with each other.

The region R7 can include a region R71 corresponding to the normal range of the vital signs information not used in the used determination method, and a region R72 and a region R73 corresponding to the abnormal ranges of the vital signs information not used in used the determination method. In the example illustrated in FIG. 5, the normal range and the abnormal range of the vital signs information not used in the used determination method are defined by a unified range U2 indicated by the unified range information generated by the controller 33 according to the above principle. Accordingly, in the region R7, a plurality of pieces of vital signs information (the graphs G4 to G7) are collectively displayed in the unified range U2. That is, in the region R7, display positions of the normal ranges of the vital signs information not used for the used determination method coincide with each other. In other words, in the region R7, the normal ranges of the vital signs information not used in the used determination method coincide with a vertical axis (an axis extending in the vertical direction in FIG. 5) in the region R7. The color tone of the region R71 is different from color tones of the regions R72 and R73. That is, the color tone related to the display mode of the normal range information is different from the color tone related to the display mode of the abnormal range information. In the present embodiment, the color tone of the region R71 is gray, and the color tone of the regions R72 and R73 is black. In the region R7, the same as or similar to the region R6, the presence or absence of dot hatching indicates that the color tone related to the display mode of the normal range information is different from the color tone related to the display mode of the abnormal range information.

In the regions R5, R6, and R7, a straight line L2 vertically crossing these regions is displayed. The straight line L2 indicates to which position of the timelines in the regions R5, R6, and R7 the specific time indicated by the selection time information (in the present embodiment, 17:52, Dec. 10, 2022) corresponds.

The region R8 below the region R7 displays the vital signs information (the respiratory information, the SpO₂ information, the body temperature information, the blood pressure information, the heart rate information, the oxygen support information, and the consciousness information) of the subject at 17:52, Dec. 10, 2022. Accordingly, the region R8 is an example of the third region.

The region R9 on a left side of the region R8 displays, as the condition information of the used determination method at 17:52, Dec. 10, 2022, the score based on the used determination method. In the present embodiment, the used determination method is qSOFA, the score based on qSOFA at 17:52, Dec. 10, 2022 is 0, and thus “0” is displayed in the region R9.

The regions R3 and R5 are located above the regions R6 and R8. Accordingly, the score (an example of the condition information) displayed in the region R3 and the bar graphs (examples of the condition information) displayed in the region R5 are displayed above the graphs G1 to G7 (that is, the vital signs information used in the used determination method and the vital signs information not used in the determination method). The graphs G1 to G3 (that is, the vital signs information used in the used determination method) are displayed above the graphs G4 to G7 (that is, the vital signs information not used in used the determination method). In addition, the score displayed in the region R3 and the bar graphs displayed in the region R5 are displayed above a portion where the graphs G1 to G7 overlap with the background information in the region R6.

Next, display data displayed on the display 42 when NEWS is used as the determination method will be described. For example, when the user types NEWS, which is the determination method indicated by the candidate determination method information, on the touch panel on the display 42 of the user terminal device 40, the display 42 displays a screen on which the display data illustrated in FIG. 6 is displayed. That is, when the user operates the user terminal device 40 to switch the determination method to be used to the determination method selected by the user, the user terminal device 40 transmits a switching request signal to the display data generation device 30. The display data generation device 30 switches the determination method to be used to the selected determination method based on the switching request signal. When the determination method indicated by the candidate determination method information is selected in this manner, the determination method indicated by the selected candidate determination method information (that is, NEWS) and the determination method indicated by the determination method information (that is, qSOFA) are switched. As illustrated in FIG. 6, contents displayed in the region R1 when NEWS is used are the same as the contents displayed in the region R1 when qSOFA is used.

Contents displayed in the region R2 when NEWS is used are similar to the contents displayed in the region R2 when qSOFA is used. However, in the example illustrated in FIG. 6, the straight line L1 is displayed below characters “NEWS”.

Contents displayed in the region R3 when NEWS is used are similar to the contents displayed in the region R3 when qSOFA is used. However, in the example illustrated in FIG. 6, the used determination method is NEWS, a score based on NEWS at 18:30, Dec. 10, 2022 is 2, and thus “2” is displayed in the region R3.

Contents displayed in region R4 when NEWS is used are the same as the contents displayed in the region R4 when qSOFA is used.

Contents displayed in the region R5 when NEWS is used are similar to the contents displayed in the region R5 when qSOFA is used. However, in the example illustrated in FIG. 6, scores based on NEWS in a period from 16:00, Dec. 10, 2022 to the current time are displayed in the region R5. In the present embodiment, when the score of NEWS is 1, a corresponding bar graph is colored in blue. When the score based on NEWS is 2, a corresponding bar graph is colored in green. When the score based on NEWS is 3 and there is no vital signs information having a score of 3 or more, a corresponding bar graph is colored in yellow. When the score based on NEWS is 4 and there is no vital signs information having a score of 3 or more, a corresponding bar graph is colored in orange. When the score based on NEWS is 5 or when there is one or more pieces of vital signs information having a score of 3 or more, a corresponding bar graph is colored in red. In FIG. 6, a bar graph hatched with oblique lines indicates a bar graph colored in blue. A bar graph hatched with vertical lines indicates a bar graph colored in green. A bar graph hatched with horizontal lines represents a bar graph colored in yellow.

Contents displayed in the region R6 when NEWS is used are similar to the contents displayed in the region R6 when qSOFA is used. However, in the example illustrated in FIG. 6, the vital signs information (the respiratory information, the SpO₂ information, the body temperature information, the blood pressure information, the heart rate information, the oxygen support information, and the consciousness information) used in NEWS from 16:00, Dec. 10, 2022 to the current time is displayed in the region R6 in a form of vital graphs extending on the timeline. The vital graph indicating the respiratory information is a graph G11. The vital graph indicating the blood pressure information is a graph G12. The vital graph indicating the consciousness information is a graph G13. The vital graph indicating the SpO₂ information is a graph G14. The vital graph indicating the body temperature information is a graph G15. The vital graph indicating the heart rate information is a graph G16. The vital graph indicating the oxygen support information is a graph G17. Also in the present embodiment, the color related to the display mode of the condition information is darker than the color related to the display mode of the background information. That is, at least one of the brightness and the chroma related to the display mode of the condition information is higher than at least one of the brightness and the chroma related to the display mode of the background information. The color related to the display mode of the background information is lighter than the colors related to the display modes of the graphs G11 to G17. That is, at least one of the brightness and the chroma related to the display mode of the background information is lower than at least one of the brightness and the chroma related to the display modes of the graphs G11 to G17.

Contents displayed in the region R7 when NEWS is used are similar to the contents displayed in the region R7 when qSOFA is used. However, since all the vital signs information (the respiratory information, the SpO₂ information, the body temperature information, the blood pressure information, the heart rate information, the oxygen support information, and the consciousness information) input to the input interface 31 in the present embodiment is vital signs information used for NEWS, no vital signs information is displayed in the region R7 in the example illustrated in FIG. 6.

Contents displayed in region R8 when NEWS is used are the same as the contents displayed in the region R8 when qSOFA is used.

Contents displayed in the region R9 when NEWS is used are similar to the contents displayed in the region R9 when qSOFA is used. However, in the example illustrated in FIG. 6, the used determination method is NEWS, a score based on NEWS at 17:52, Dec. 10, 2022 is 3, and thus “3” is displayed in the region R3.

There is a need to check vital signs information of a subject and condition information indicating a condition of the subject at a glance. At this time, it is desired that a condition level of the subject indicated by the condition information can also be checked at a glance. For example, in a known patient monitor, a trend graph of vital signs information and a trend graph of an alarm setting value can be checked on the same screen, but a location where an alarm occurs can be recognized only in the trend graph of the alarm setting value. In this respect, there is room for improvement in the known patient monitor.

According to the display data generation device 30 having the above configuration, display data including vital signs information and background information is generated, in which the background information overlaps with the vital signs information. The background information indicates the background corresponding to the condition of the subject, and is thus information indicating the condition of the subject. Accordingly, the display data generated by the display data generation device 30 can include not only the vital signs information but also the background information that indicates the condition of the subject and overlaps with the vital signs information. For this reason, the user can visually recognize the vital signs information and the background information overlapping with the vital signs information at the same time, so that the condition of the subject can be checked at a glance. According to the display data generation device 30, the vital signs information of the subject and the information indicating the condition of the subject can be checked at a glance. A display data generation method, a computer program, and a non-transitory computer-readable storage medium according to the above configuration can also implement the same or similar effect.

According to the display data generation device 30 having the above configuration, since the color tone related to the display mode of the background information is different from the color tone related to the display mode of the vital signs information, the user can easily distinguish and visually recognize the background information and the vital signs information.

According to the display data generation device 30 having the above configuration, the color related to the display mode of the background information is lighter than the color related to the display mode of the vital signs information. According to the display data generation device 30, the visibility of the vital signs information is not impaired even when the background information overlaps with the vital signs information.

According to the display data generation device 30 having the above configuration, the display data can further include the normal range information and the abnormal range information, and the vital signs information overlaps with the background information and at least one of the normal range information and the abnormal range information. That is, the vital signs information overlaps with not only the background information but also at least one of the normal range information and the abnormal range information. Accordingly, the user can easily recognize the condition of the subject and whether the vital signs information of the subject is within the normal range.

According to the display data generation device 30 having the above configuration, the color tone related to the display mode of the normal range information is different from the color tone related to the display mode of the abnormal range information. Accordingly, the user who visually recognizes the display data can easily distinguish between the normal range and the abnormal range.

According to the display data generation device 30 having the above configuration, in the display data, a plurality of pieces of vital signs information are collectively displayed in the unified range based on the normal range information and the abnormal range information corresponding to the vital signs information. That is, even when the display data can include a plurality of pieces of vital signs information having different normal range information and different abnormal range information, the plurality of pieces of vital signs information are collectively displayed in the unified range. For this reason, according to the display data generation device 30, the user can visually recognize a plurality of pieces of vital signs information at a glance, and can easily recognize whether the plurality of pieces of vital signs information are within the normal range. Further, according to the display data generation device 30, the user can easily visually recognize a plurality of pieces of vital signs information with a small amount of viewpoint movement, and thus a sense of burden on the user can be reduced.

According to the display data generation device 30 having the above configuration, the vital graphs of the vital signs information extending on the timeline are also displayed in the display data. Accordingly, not only the condition of the subject but also a change over time in the vital signs information of the subject can be checked.

According to the display data generation device 30 having the above configuration, the display data can include the condition information in addition to the background information. Since the condition information and the background information are displayed in correspondence along the timeline, the user can more reliably grasp the condition of the subject by, for example, visually recognizing the condition information that is more easily visually recognized than the background information.

According to the display data generation device 30 having the above configuration, the condition information does not overlap with the vital signs information, and the user can easily visually recognize the vital signs information and the condition information. Accordingly, the user can more reliably grasp the condition of the subject.

According to the display data generation device 30 having the above configuration, the hue related to the display mode of the condition information corresponds to the hue related to the display mode of the background information. Accordingly, the user can intuitively recognize that the condition information and the background information correspond to each other.

According to the display data generation device 30 having the above configuration, the color related to the display mode of the condition information is darker than the color related to the display mode of the background information, and thus the condition information is more visibly recognized by the user than the background information is. For this reason, the user can easily visually recognize the condition information, and can thus quickly grasp the condition of the subject.

According to the display data generation device 30 having the above configuration, at least one of the brightness and the chroma related to the display mode of the condition information is higher than at least one of the brightness and the chroma related to the display mode of the background information, and thus the user can reliably grasp the condition of the subject.

According to the display data generation device 30 having the above configuration, the condition information is displayed above the portion where the vital signs information overlaps with the background information, and thus the user can reliably grasp the condition of the subject.

### Second Embodiment

Next, a display data generation method according to the present embodiment will be described with reference to FIGS. 4 and 7. In the present embodiment, the same or similar parts as those in the first embodiment are denoted by the same or similar reference numerals, and detailed description thereof will be appropriately omitted. Also in the present embodiment, the display data generation method illustrated in FIG. 4 is executed. Also in the present embodiment, a current time is assumed to be 18:30, Dec. 10, 2022. Also in the present embodiment, it is assumed that vital signs information is obtained from 16:00 to 18:30, Dec. 10, 2022.

STEP 01 to STEP 06 are the same as or similar to those in the first embodiment. In STEP 04, it is assumed that the user inputs determination method information indicating that the used determination method is qSOFA to the input 41 of the user terminal device 40.

The controller 33 generates the unified range information for the vital signs information (the SpO₂ information, the body temperature information, the heart rate information, and the oxygen support information) not used for qSOFA (STEP 08). In the present embodiment, no unified range information is generated for the vital signs information (the respiratory information, the blood pressure information, and the consciousness information) used for qSOFA. A method for generating the unified range information in the present embodiment is the same as or similar to the method for generating the unified range information in the first embodiment, and a detailed description thereof will be omitted.

STEP 09 to STEP 14 are the same as or similar to those in the first embodiment. In STEP 10, it is assumed that the user inputs, to the input 41 of the user terminal device 40, selection time information indicating that the time at which the user desires to check the vital signs information is 17:52, Dec. 10, 2022.

Here, the display data displayed on the display 42 of the user terminal device 40 in STEP 14 will be described. In the present embodiment, the display data displayed on the display 42 of the user terminal device 40 is the display data illustrated in FIG. 7. The contents displayed in the regions R1 to R5 and the regions R7 to R9 in the present embodiment are the same as or similar to the contents displayed in the regions R1 to R5 and the regions R7 to R9 in the first embodiment. On the other hand, in the display data in the present embodiment, regions R6A to R6C are displayed instead of the region R6. The region R6A displays a vital graph G21 of the respiratory information. The region R6B displays a vital graph G22 of the blood pressure information. The region R6A displays a vital graph G23 of the consciousness information. The regions R6A, R6B, and R6C are examples of a first region. In this manner, the present embodiment is different from the first embodiment in that the vital signs information not used in the used determination method is collectively displayed in the unified range U2, and the vital signs information used in the used determination method is separately displayed (that is, not collectively displayed in the unified range).

Also in the second embodiment, the display data generation device 30 can implement the same or similar effects as those of the first embodiment.

The above-described embodiments are for facilitating understanding of the presently disclosed subject matter, and do not limit the presently disclosed subject matter. The presently disclosed subject matter can be modified or improved without departing from the gist thereof.

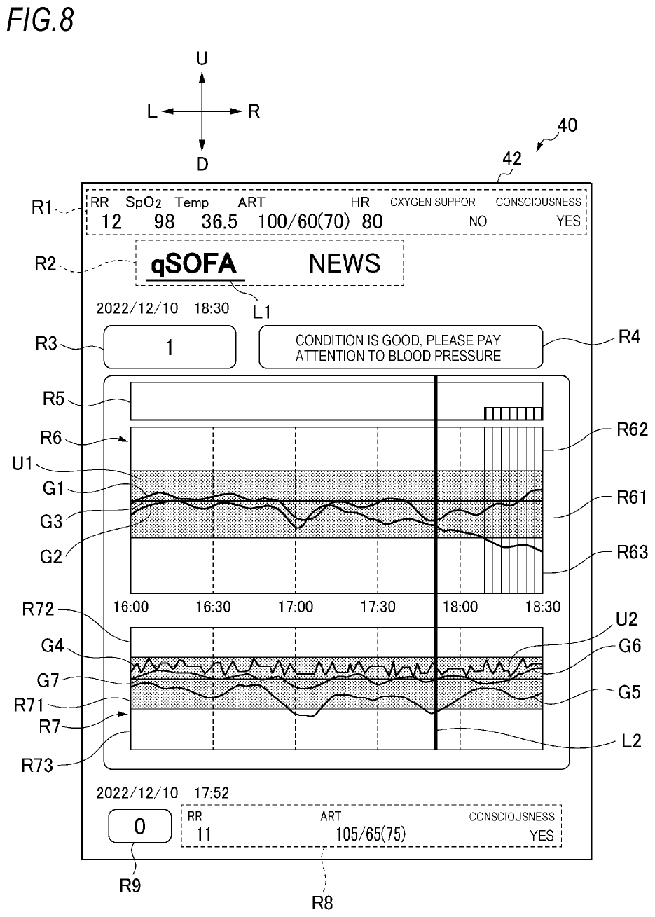

In the above embodiments, the region R8 (an example of the third region) displays the respiratory information, the SpO₂ information, the body temperature information, the blood pressure information, the heart rate information, the oxygen support information, and the consciousness information obtained at 17:52, Dec. 10, 2022 (an example of the specific time freely selected by the user), and the presently disclosed subject matter is not limited to this example. For example, the region R8 may display only the vital signs information used for the used determination method among the vital signs information obtained at the specific time freely selected by the user. For example, when the used determination method is qSOFA, the display data displayed on the display 42 of the user terminal device 40 is the display data illustrated in FIG. 8. The display data illustrated in FIG. 8 is different from the display data illustrated in FIG. 5 in that only the respiratory information, the blood pressure information, and the consciousness information, which are vital signs information used in qSOFA, are displayed in the region R8. When the display data generation device 30 generates the display data illustrated in FIG. 8, only the vital signs information used for the determination method for determining the condition of the subject among the vital signs information obtained at the specific time is displayed in the region R8. For this reason, the user can quickly recognize the vital signs information used in the determination method by visually recognizing the vital signs information displayed in the region R8.

In the above embodiments, the display data is displayed on the display 42 of the user terminal device 40, and may be displayed on the display 34 of the display data generation device 30 or the display 24 of the bedside monitor 20, for example.

In the above embodiments, the display data can include the vital signs information, the background information, the normal range information, the abnormal range information, the condition information, the determination method information, the candidate determination method information, and the identification information. Alternatively, the presently disclosed subject matter is not limited these examples. For example, the display data may not include the normal range information, the abnormal range information, the condition information, the determination method information, the candidate determination method information, or the identification information.

In the above embodiments, a plurality of pieces of vital signs information are input to the input interface 31. Alternatively, one piece of vital signs information may be input.

In the second embodiment, the vital signs information used in the used determination method is separately displayed, and the vital signs information not used in the determination method is collectively displayed in the unified range U2. For example, all the obtained vital signs information (that is, the vital signs information used in the used determination method and the vital signs information not used in the determination method) may be individually displayed in separate regions. In addition, for example, the vital signs information used in the used determination method may be collectively displayed in the unified range U1, and the vital signs information not used in the determination method may be displayed in individually separated regions.

In the above embodiments, the respiratory information, the SpO₂ information, the body temperature information, the blood pressure information, the heart rate information, the oxygen support information, and the consciousness information are input to the input interface 31. Alternatively, for example, vital signs information used for the determination method alone may be input.

In the first embodiment, for example, when the used determination method is qSOFA, three pieces of vital signs information are displayed in the unified range U1 of the region R6, and four pieces of vital signs information are displayed in the unified range U2 of the region R7. Alternatively, the presently disclosed subject matter is not limited thereto, and the number of pieces of vital signs information may be two or more.

In the above embodiments, two determination methods (qSOFA and NEWS) are displayed in the region R2. Alternatively, for example, three or more determination methods may be displayed.

In the above embodiments, the display data generation system 1 can include the sensor 10, the bedside monitor 20, the display data generation device 30, and the user terminal device 40. Alternatively, the display data generation system 1 may not include the display data generation device 30 or the user terminal device 40. In this case, the bedside monitor 20 may include a storage and a controller 22 corresponding to the storage 32 and the controller 33 of the display data generation device 30, and the bedside monitor 20 may implement functions of the display data generation device 30. The display data generation system 1 may not include the bedside monitor 20 or the display data generation device 30. In this case, the user terminal device 40 may include a storage and a controller corresponding to the storage 32 and the controller 33 of the display data generation device 30, and the user terminal device 40 may implement functions of the display data generation device 30.

In the above embodiments, the display data is generated by the display data generation device 30, and may also be generated by the bedside monitor 20 or the user terminal device 40. In this case, the bedside monitor 20 or the user terminal device 40 can include, for example, a controller corresponding to the display data generation device 30, and obtains various types of information from the storage 32 of the display data generation device 30.

As described above, the following matters are disclosed in the present specification.
(1) A display data generation device including:
   an input interface configured to receive vital signs information of a subject;
   one or more processors; and
   one or more memories configured to store at least one instruction executable by the processor, wherein
   when the at least one command is executed by the processor, the display data generation device generates display data, the display data including the vital signs information and background information that indicates a background corresponding to a condition of the subject determined based on the vital signs information and a determination method for determining the condition of the subject, the background information overlapping with the vital signs information.
(2) The display data generation device according to (1), wherein
   a color tone related to a display mode of the background information is different from a color tone related to a display mode of the vital signs information.
(3) The display data generation device according to (2), wherein
   a color related to the display mode of the background information is lighter than a color related to the display mode of the vital signs information.
(4) The display data generation device according to any one of (1) to (3), wherein
   the display data further includes normal range information indicating a normal range of the vital signs information that is set for each piece of the vital signs information and abnormal range information indicating an abnormal range of the vital signs information that is set for each piece of the vital signs information, and
   the vital signs information overlaps with the background information and at least one of the normal range information and the abnormal range information.
(5) The display data generation device according to (4), wherein
   a color tone related to a display mode of the normal range information is different from a color tone related to a display mode of the abnormal range information.
(6) The display data generation device according to (4) or (5), wherein
   the display data generation device generates the display data in which a plurality of pieces of the vital signs information received by the input interface are collectively displayed in a unified range, the unified range being based on the normal range information and the abnormal range information corresponding to each piece of the vital signs information, the unified range being used to uniformly define normal ranges and abnormal ranges of the plurality of pieces of vital signs information.
(7) The display data generation device according to any one of (1) to (6), wherein
   a vital graph of the vital signs information extending on a timeline is displayed in the display data.
(8) The display data generation device according to any one of (1) to (7), wherein
   the display data further includes condition information that indicates a condition of the subject and is generated based on the vital signs information and the determination method,
   the condition information does not overlap with the vital signs information, and
   the condition information and the background information are displayed in correspondence along a timeline.
(9) The display data generation device according to (8), wherein
   the condition information does not overlap with the vital signs information.
(10) The display data generation device according to (9), wherein
   a hue related to a display mode of the condition information corresponds to a hue related to the display mode of the background information.
(11) The display data generation device according to (10), wherein
   a color related to the display mode of the condition information is darker than a color related to the display mode of the background information.
(12) The display data generation device according to any one of (8) to (11), wherein
   the condition information is displayed above a portion where the vital signs information overlaps with the background information.
(13) A display data generation method executed by a display data generation device, the method including:
   generating display data, the display data including vital signs information of a subject and background information that indicates a background corresponding to a condition of the subject determined based on the vital signs information and a determination method for determining the condition of the subject, the background information overlapping with the vital signs information.
(14) A non-transitory computer-readable storage medium storing a computer program executable by one or more processors provided in a display data generation device, when being executed, the computer program causing the display data generation device to
   generate display data, the display data including vital signs information of a subject and background information that indicates a background corresponding to a condition of the subject determined based on the vital signs information and a determination method for determining the condition of the subject, the background information overlapping with the vital signs information.

What is claimed is:

1. A display data generation device comprising:
an input interface configured to receive vital signs information of a subject;
one or more processors; and
one or more memories configured to store at least one instruction executable by the processor, wherein
when the at least one command is executed by the processor, the display data generation device generates display data, the display data including the vital signs information and background information that indicates a background corresponding to a condition of the subject determined based on the vital signs information and a determination method for determining the condition of the subject, the background information overlapping with the vital signs information.

2. The display data generation device according to claim 1, wherein
a color tone related to a display mode of the background information is different from a color tone related to a display mode of the vital signs information.

3. The display data generation device according to claim 2, wherein
a color related to the display mode of the background information is lighter than a color related to the display mode of the vital signs information.

4. The display data generation device according to claim 1, wherein
the display data further includes normal range information indicating a normal range of the vital signs information that is set for each piece of the vital signs information and abnormal range information indicating an abnormal range of the vital signs information that is set for each piece of the vital signs information, and
the vital signs information overlaps with the background information and at least one of the normal range information and the abnormal range information.

5. The display data generation device according to claim 4, wherein
a color tone related to a display mode of the normal range information is different from a color tone related to a display mode of the abnormal range information.

6. The display data generation device according to claim 4, wherein
the display data generation device generates the display data in which a plurality of pieces of the vital signs information received by the input interface are collectively displayed in a unified range, the unified range being based on the normal range information and the abnormal range information corresponding to each piece of the vital signs information, the unified range being used to uniformly define normal ranges and abnormal ranges of the plurality of pieces of vital signs information.

7. The display data generation device according to claim 1, wherein
a vital graph of the vital signs information extending on a timeline is displayed in the display data.

8. The display data generation device according to claim 1, wherein
the display data further includes condition information that indicates a condition of the subject and is generated based on the vital signs information and the determination method, and
the condition information and the background information are displayed in correspondence along a timeline.

9. The display data generation device according to claim 8, wherein
the condition information does not overlap with the vital signs information.

10. The display data generation device according to claim 9, wherein
a hue related to a display mode of the condition information corresponds to a hue related to the display mode of the background information.

11. The display data generation device according to claim 10, wherein
a color related to the display mode of the condition information is darker than a color related to the display mode of the background information.

12. The display data generation device according to claim 8, wherein
the condition information is displayed above a portion where the vital signs information overlaps with the background information.

13. A display data generation method executed by a display data generation device, the method comprising:
generating display data, the display data including vital signs information of a subject and background information that indicates a background corresponding to a condition of the subject determined based on the vital signs information and a determination method for determining the condition of the subject, the background information overlapping with the vital signs information.

14. A non-transitory computer-readable storage medium storing a computer program executable by one or more processors provided in a display data generation device, when being executed, the computer program causing the display data generation device to
generate display data, the display data including vital signs information of a subject and background information that indicates a background corresponding to a condition of the subject determined based on the vital signs information and a determination method for determining the condition of the subject, the background information overlapping with the vital signs information.
